SEQUENCE LISTING (18 sequences)

SEQ ID NO: 1 - DNA, 1029 bp, Burkholderia pseudomallei
atgaaggatacgcagctcaacgtctataactggtcggactacattgcgaaggacacgatcccgaacttcgagaagcagacgggcgtcaaggtccgctacgacaactacgacagcgacgacacgctgcaggcgaagcttttgaccggcaactcgggctacgacatcgtcgtgccgacgagcaactacgcgggcaagcagatccaggccggcatcttcacgccgctcgacaagtcgaagctgccgaacctcaagtatctcgatgcgcaactgatggcgctcgtcgcgggcgccgatccgggcaacaagtacgtggtgccctgggcgtatggcacgacgggcctcggctacaacgtcgacaaggcgcagaaggtgctcggcaaggtgccgctcgacaactgggacatcctgttcaagccggagaacctctcgaagctgaagacgtgcggcgtgtcggtgctcgacgcgccggaccagatgttcgcggcgacgctgcactacatcggcaaggatccgatgagcacgaacccggccgactaccaggccgcgatgcaggtgctcaagaagatccgcccgtacatcacgcagttcaactcgtcgggctacatcaacgacatggtcggcggcgacatctgcttcgcgttcggctggtcgggcgacgtcgtgatcgcgaagcaccgcgcgctcgaggcgaagaagccgtacaagctcgagtactacgtgccgaagggcggcgcgcccgtatggttcgacgtgatggcgatcccgaaggacgcgaagaacaaggacgccgcgcttcaatggatcaactacatcgaggatccgaaggttcacgcgtcgatcacgaacgcggtctactacccgagcgcgaacgcgcaagcgcgcaagtacgtgcgcccggatgtcgcgaacgacccggccgtgtacccgcctccggacgtcgtgaagacgctgttcctgctcaagccgctgccgcccgagatccagcgcctgcagacgcgtctttggaccgagctgaaatcgggacgctga

SEQ ID NO: 2 - DNA, 1539 bp, Burkholderia pseudomallei
atgtcgaatgtcacgctcgcggatcagcaggacctcacgcggcaggtgcccgcggaggtcgaatcgctcgaccccgcgcacatcgaatcgtggaccggcaacacgatcggcctggatctgttcgaggggctcgcgcgcatcgacgcgagcggggcggtggtgccgggcgtcgcgcaggcgtgggagcacaaggcgccggatacgtggatcttcaagctgcgccgcgacgcgaagtggagcaacgggcagcccgtgacggccgcggatttcgtatacgcgtggcagcgtctcgccgatccgaagacgggttcgaaatacacgatcctcgtcgagttcgtgaagaacgcctcggcgatcatcgcgggcaagcagccgcccggcgatctcggcattcgcgcgatcgatccgtacacgatcgaggtgaagaccgaggtgcccgtgtcgtatttccccgagctcaccgcgatggcgccgctcacccccgtgaacaaggacgcggtcgcgaagttcggcgacgcatggacgcgcccgaagaacatcgtgagcaacggcccttacacgctcgtcgactggcagccgaacaaccggatcgtgatggcgaagagcgacaagtactggaacgcgcgcaacgtcgtgatccgcaaggtgacgtacctgccgatcgagaacgacgagaccgcgctgcggatgtaccaggcgggccagatcgactacacgtattcgattcccgcgggcggcttcggccagatcagcaagcagttcggcaaggagctgcgcccggggctgcagctcgcgacgtattactactacctgaagaacagcgatccggcgctcaaggacaagcgcgtgcgcgaagcgctcgcgatggtgctcgaccgcgagatcctcacgtcgaagatcacgcaggcgggcgaagtgccgatgtacgggctgatgccgaagggcgtgaagggcgtgcagcggccgttcacgccggactgggcgtcgtggccgatggcgaggcgcgtcgactacgcgaagaacctgctgaagcaggcgggccatggcgacgcgaatccgctcacgttcacgctgacctacaacacgaacgacctgcacaagaaggtcgcgctgttcgcggcgtccgaatggcgcacgaagctcggcgtgacggcgaagctcgagaacgtcgagttcaaggtgctgatgaagcagcgccacgacggcaaggtgcagatcgcgcgcgacggctggttcgccgactacaacgacgcgatgacgttcttcgacctgatccgctgcggcagctcgcagaacacggtcggctactgcaacccgaaggtcgattcgctcgtcgccgaggcgaaccagaagctcgatgacggcgcgcgcgcggcgctgctcacgcaggcgcacgatctcgcgatgaacgactatccgatggtgccgctcttccagtattcggcggaccgcctcgtgaagtcgtacgtcggcggctacacgctgacgaactacatcgacatgcgcgcctcgcaggacatgtacctgatcaagcactga

SEQ ID NO: 3 - DNA, 687 bp, Burkholderia pseudomallei
atggtgctgtcggtgatgaacggcttccagaaagaggtgcgcgaccggatgctgtcggtgctcgcgcacgtcgagatcttctcgccgacgggctcgatgcccgactggcaactgaccgcgaaggaagcgcgcctgaaccgctcggtgatcggcgcggcgccgtatgtcgacgcgcaggcgctgctcacgcggcaggacgcggtgagcggcgtgatgctgcgcggcgtcgagccgtcgctcgagccgcaggtgtccgacatcggcaaggacatgaaggcgggcgcgctcaccgcgctcgcgcccggccagttcgggatcgtgctcggcaacgcgctcgcgggcaatctcggcgtgggcgtcggcgacaaggtcacgctcgtcgcgccggaaggcacgatcacgccggccgggatgatgccgcgcctgaagcagttcacggtggtcggcatcttcgagtccgggcactacgaatacgacagcacgcttgcgatgatcgacatccaggatgcgcaggcgctcttcaggctgcccgcgccgacgggcgtgcggctgcgcctcacggacatgcagaaggcgccgcaggtcgcgcgcgagctcgcgcacacgctgtcgggcgacctgtacatccgcgactggacgcagcagaacaagacgtggttctccgcggtgcagatcgagaagcggtga

SEQ ID NO: 4 - protein, 227 aa, Burkholderia pseudomallei
VLSVMNGFQKEVRDRMLSVLAHVEIFSPTGSMPDWQLTAKEARLNRSVIGAAPYVDAQALLTRQDAVSGVMLRGVEPSLEPQVSDIGKDMKAGALTALAPGQFGIVLGNALAGNLGVGVGDKVTLVAPEGTITPAGMMPRLKQFTVVGIFESGHYEYDSTLAMIDIQDAQALFRLPAPTGVRLRLTDMQKAPQVARELAHTLSGDLYIRDWTQQNKTWFSAVQIEKR

SEQ ID NO: 5 - protein, 341 aa, Burkholderia pseudomallei
KDTQLNVYNWSDYIAKDTIPNFEKQTGVKVRYDNYDSDDTLQAKLLTGNSGYDIVVPTSNYAGKQIQAGIFTPLDKSKLPNLKYLDAQLMALVAGADPGNKYVVPWAYGTTGLGYNVDKAQKVLGKVPLDNWDILFKPENLSKLKTCGVSVLDAPDQMFAATLHYIGKDPMSTNPADYQAAMQVLKKIRPYITQFNSSGYINDMVGGDICFAFGWSGDVVIAKHRALEAKKPYKLEYYVPKGGAPVWFDVMAIPKDAKNKDAALQWINYIEDPKVHASITNAVYYPSANAQARKYVRPDVANDPAVYPPPDVVKTLFLLKPLPPEIQRLQTRLWTELKSGR

SEQ ID NO: 6 - protein, 511 aa, Burkholderia pseudomallei
SNVTLADQQDLTRQVPAEVESLDPAHIESWTGNTIGLDLFEGLARIDASGAVVPGVAQAWEHKAPDTWIFKLRRDAKWSNGQPVTAADFVYAWQRLADPKTGSKYTILVEFVKNASAIIAGKQPPGDLGIRAIDPYTIEVKTEVPVSYFPELTAMAPLTPVNKDAVAKFGDAWTRPKNIVSNGPYTLVDWQPNNRIVMAKSDKYWNARNVVIRKVTYLPIENDETALRMYQAGQIDYTYSIPAGGFGQISKQFGKELRPGLQLATYYYYLKNSDPALKDKRVREALAMVLDREILTSKITQAGEVPMYGLMPKGVKGVQRPFTPDWASWPMARRVDYAKNLLKQAGHGDANPLTFTLTYNTNDLHKKVALFAASEWRTKLGVTAKLENVEFKVLMKQRHDGKVQIARDGWFADYNDAMTFFDLIRCGSSQNTVGYCNPKVDSLVAEANQKLDDGARAALLTQAHDLAMNDYPMVPLFQYSADRLVKSYVGGYTLTNYIDMRASQDMYLIKH

SEQ ID NO: 7 - DNA, 18 bp, Artificial sequence
  Synthetic sequence Oligonucleotide primer
cggccgcggatttcgtat

SEQ ID NO: 8 - DNA, 21 bp, Artificial sequence
  Synthetic sequence Oligonucleotide primer
gcagcgcggtctcgtcgttct

SEQ ID NO: 9 - DNA, 19 bp, Artificial sequence
  Synthetic sequence Oligonucleotide primer
atccaggccggcatcttca

SEQ ID NO: 10 - DNA, 18 bp, Artificial sequence
  Synthetic sequence Oligonucleotide primer
agcgcggcgtccttgttc

SEQ ID NO: 11 - DNA, 24 bp, Artificial sequence
  Synthetic sequence Oligonucleotide primer
aactttgagggcatgcttaccgtc

SEQ ID NO: 12 - DNA, 24 bp, Artificial sequence
  Synthetic sequence Oligonucleotide primer
tcattcatagcgcaccgcctccgc

SEQ ID NO: 13 - DNA, 27 bp, Artificial sequence
  Synthetic sequence Oligonucleotide primer
aggatcctaatgtcgaatgtcacgctc

SEQ ID NO: 14 - DNA, 27 bp, Artificial sequence
  Synthetic sequence Oligonucleotide primer
aaagctttattcagtgcttgatcaggt

SEQ ID NO: 15 - DNA, 27 bp, Artificial sequence
  Synthetic sequence Oligonucleotide primer
agaattctaatgaaggatacgcagctc

SEQ ID NO: 16 - DNA, 30 bp, Artificial sequence
  Synthetic sequence Oligonucleotide primer
agcggccgctattcagcgtcccgatttcag

SEQ ID NO: 17 - DNA, 27 bp, Artificial sequence
  Synthetic sequence Oligonucleotide primer
aggatcctaatggtgctgtaggtgatg

SEQ ID NO: 18 - DNA, 27 bp, Artificial sequence
  Synthetic sequence Oligonucleotide primer
aaagctttattcaccgcttctcgatct

(19) United States
(12) Patent Application Publication
(10) Pub. No.: US 2010/0055123 A1
(43) Pub. Date: Mar. 4, 2010

(54) VACCINE AGAINST BURKHOLDERIA INFECTIONS

(76) Inventors: David Neil Harland, Salisbury (GB); Helen Susan Atkins, Salisbury (GB); Nicola Jane Walker, Salisbury (GB); Michelle Nelson, Salisbury (GB); Richard William Titball, Exeter (GB); Timothy Phillip Atkins, Salisbury (GB)

Correspondence Address: JOHN S. PRATT, ESQ;KILPATRICK STOCKTON, LLP, 1100 PEACHTREE STREET, SUITE 2800, ATLANTA, GA, 30309, US

(21) Appl. No.: 11/911,541

(22) PCT Filed: Apr. 13, 2006
(86) PCT No.: PCT/GB2006/001354

§ 371 Date: May 18, 2009

(30) Foreign Application Priority Data
Apr. 15, 2005 (GB) 0507632.8

(51) Int. Cl. A61K 39/02; A61K 38/16; C07K 14/195; C07K 16/12; C07H 21/04

(52) U.S. Cl. 424/190.1; 514/12; 530/350; 530/387.9; 536/23.7

(57) ABSTRACT

This invention relates to proteins expressed by ABC system genes of Burkholderia pseudomallei. The proteins (or protective fragments or protective variants thereof) are protective against Burkholderia pseudomallei infection. Use of the proteins in treating Burkholderia mallei or Burkholderia pseudomallei infection and uses in the preparation of vaccines against melioidosis and glanders are described and claimed.

This invention relates to ATP-binding cassette (ABC) system proteins derived from Burkholderia pseudomallei, to their use in producing a protective immune response in mammals and to the preparation of vaccines against melioidosis and glanders. The invention also provides a method for the production of such proteins.

Burkholderia pseudomallei is a saprophytic, Gram negative, non-spore forming bacillus most commonly isolated in South East Asia. In this region and particularly in Thailand, Vietnam and Northern Australia, B. pseudomallei is a major cause of morbidity and mortality as the causative agent of the emerging infection melioidosis. Melioidosis has four different forms of disease: acute, sub-acute, chronic and sub-clinical. The acute, sub acute and chronic infections progress systemically giving rise to septicaemic disease, which in untreated cases has a mortality rate of 80-90% with death occurring within 24-48 hours of the onset of symptoms. Acute septicaemia is usually associated with patients with an existing immunodeficiency and in many cases adult melioidosis is the result of re-infection or re-activation of latent infections, usually contracted in early in life in endemic areas. There is currently no licensed vaccine for melioidosis. The primary treatment regime for melioidosis consists of antibiotic treatment with intravenous ceftazidime for 10-14 days. However, B. pseudomallei is amongst the growing number of bacterial pathogens that exhibit an increasingly high level of antibiotic resistance and as a result the need for an effective vaccine is pressing. To date, no proteins have been identified as protective against melioidosis.

ABC systems are responsible for the transport of a wide variety of different molecules across cellular membranes. The ABC systems constitute one of the largest protein superfamilies and are ubiquitous in nature, being found in bacteria, eukarya and archea (C. F. Higgins, Annu. Rev. Cell Biol. 8 (1992) 67-113). Bacterial ABC transporters are extremely versatile transport systems which vary greatly in size and function to either import or export a range of substances denoted as ‘allocrites’. Due to the diverse nature of the allocrites, these transport systems have been shown to play different roles in many cellular functions including bacterial metabolism and virulence. For example, importers are often involved in the uptake of nutrients such as sugars, iron and peptides, and are therefore important in bacterial metabolism. Furthermore, inactivation of ABC import systems can lead to attenuation. For example, inactivation of the sfbABC iron uptake system in Salmonella enteritidis results in attenuation in mice. In comparison, ABC transporters that function to export allocrites are often more directly associated with virulence and pathogenicity, either directly through the export of toxins such as haemolysin or by improving survival through antibiotic resistance.

The common feature of ABC systems is the presence of protein components containing highly conserved ATP-binding cassettes (ABCs) that are responsible for the generation of energy for the transport of allocrites through the hydrolysis of ATP to ADP. ABCs include Walker A and Walker B sites for binding ATP, a number of membrane spanning domains (MSDs), and a specific signature sequence. The normal structural arrangement within ABC proteins is a four domain arrangement—two domains containing ABCs and two domains containing MSDs arranged on one or more polypeptide chains. Other proteins may also be involved in import and export of allocrites in ABC systems. For example, periplasmic solute binding proteins are usually involved in import in Gram negative organisms. Additional outer membrane proteins such as TolC may be required for the export of allocrites.

A number of ABC system proteins have been identified as immunogenic, including predicted solute binding proteins and ABC containing proteins, usually identified through a process of screening anti-serum raised against whole bacterial cells. This identification of immunogenic ABC system proteins, coupled with additional evidence of the ability of ABC system proteins to protect against Brucella abortus, Mycobacterium tuberculosis and Streptococcus pneumoniae infection, has prompted the assertion that ABC system proteins may constitute potential vaccine antigens (see, for example, Garmory, R. S. & Titball, R. W., Infection and Immunity, 72 (2004), 6757-6763). However, despite the fact that this has led to the targeting of such proteins as potential vaccine antigens, it has been found that many, if not most, of the ABC system proteins isolated to date are largely ineffective as vaccine antigens, irrespective of their ability to elicit an immune response (see, for example, Atkins, H. S. et al, Research in Microbiology, 2006, in press). Certainly, there is no indication in the prior art that ABC system proteins, in particular those derived from Burkholderia species, may be used as protective antigens.

The applicants have now identified several proteins which are derived from the ABC systems of Burkholderia pseudomallei and which produce a protective immunogenic response against Burkholderia infection, when administered to a mammal. The proteins have been cloned, expressed and purified and their potential to induce protective immunity against Burkholderia pseudomallei tested in the mouse model of infection. Consequently, these proteins are useful as vaccines against diseases caused by Burkholderia infections, such as Melioidosis and Glanders.

Specifically, these proteins are derived from ABC systems of Burkholderia pseudomallei and include proteins which are predicted to be present in the region of the periplasmic space and outer membrane areas of the bacterium, such as periplasmic binding proteins, and also inner membrane proteins which are thought to be involved in export processes. The proteins are capable of producing a protective immune response against Burkholderia pseudomallei and Burkholderia mallei and, as such, fragments of the proteins which are capable of providing a protective immune response and variants of the proteins which are capable of providing a protective immune response are also included within the scope of the invention.

As used herein, the term “protein” means a sequence of amino acids joined together by peptide bonds. The amino acid sequence of the protein is determined by the sequence of DNA bases which encode the amino acids of the protein. As used herein protein includes polypeptides and herein, the terms “protein” and “polypeptide” are used interchangeably.

According to the present invention there is provided a protein expressed by an ABC system gene of Burkholderia pseudomallei or a fragment or a variant of said protein, wherein the protein, fragment or variant is capable of producing a protective immune response against Burkholderia mallei or Burkholderia pseudomallei infection, for use as a medicament.

In particular, the protein is for use as a prophylactic or therapeutic vaccine against infection by Burkholderia mallei and Burkholderia pseudomallei. Prophylactic vaccines are particularly preferred.

In the context of the present invention the expression “variant” as used herein refers to sequences of amino acids which differ from the base sequence from which they are derived in that one or more amino acids within the sequence are substituted for other amino acids. Amino acid substitutions may be regarded as “conservative” where an amino acid is replaced with a different amino acid with broadly similar properties. “Non-conservative” substitutions are where amino acids are replaced with amino acids of a different type. Broadly speaking, fewer non-conservative substitutions will be possible without altering the biological activity of the polypeptide. Suitably variants will be at least 80% identical, preferably at least 90% identical, and more preferably at least 95% identical to the base sequence.

Identity in this instance can be judged, for example, using the BLAST program (version 2.2.12) found at http:www.ncbi.nim.nih.gov/BLAST or by using the algorithm of Lipman-Pearson, with Ktuple: 2, gap penalty: 4, Gap Length Penalty: 12, standard PAM scoring matrix (Lipman, D. J. and Pearson W. R., Rapid and Sensitive Protein Similarity Searches, Science, 1985, vol. 227, 1435-1441).

The term “fragment” refers to any portion of the given amino acid sequence of a polypeptide which has the same activity as the complete amino acid sequence. Fragments will suitably comprise at least 5 and preferably at least 10 consecutive amino acids from the basic sequence, or combinations of these fragments. In order to retain activity, fragments will suitably comprise at least one epitopic region. Fragments comprising epitopic regions may suitably be fused together to form variants within the above definition.

The phrase “producing a protective immune response” as used herein means that the substance is capable of generating a protective immune response in a host organism, such as a mammal, for example, a human, to whom it is administered.

Proteins of the invention are further characterised in that they bind directly to, or are components of systems that bind to adenosine triphosphate (ATP). Examples of such proteins include periplasmic binding proteins such as OppA (CDS no. YP 112142) and PotF (CDS no. YP 106734), and inner membrane export/releasing proteins such as “LolC” obtained from Burkholderia pseudomallei. Further examples of such proteins include fragments or variants of OppA, PotF or LolC as described above.

In particular it has been found that polypeptides with any of SEQ ID Nos 4, 5 or 6 are preferred examples of proteins of the invention. A more preferred example is the polypeptide with SEQ ID no4. Therefore this protein and protective fragments and variants thereof form a particularly preferred embodiment of the invention.

Proteins as described above may be prepared using methods according to another aspect of the invention. Such methods comprise firstly, identifying the ABC system genes in the genome of the Burkholderia species, for example the Burkholderia pseudomallei K96243 genome sequence. ABC system genes may be identified by scanning the genome for ABC system signature sequences, such as PS00211, using any suitable commercially available genome viewing software, such as Artemis. Once the ABC system proteins have been identified, the genes that encode the proteins, or the fragments of the genes that encode specific peptide regions of the corresponding ABC system proteins are amplified. The amplified gene fragments may then be cloned into an expression vector to produce a recombinant plasmid, which may, in turn be transformed into a host cell. Host cells may be prokaryotic or eukaryotic cells. It is preferred that the host cell is a prokaryotic cell, such as Escherichia coli.

According to a second aspect of the invention there is provided a pharmaceutical composition comprising a protein expressed by an ABC system gene of Burkholderia pseudomallei or a fragment or a variant of said protein, wherein the protein, fragment, or variant is capable of producing a protective immunogenic response against Burkholderia mallei or Burkholderia pseudomallei, in combination with a pharmaceutically acceptable carrier or excipient.

Suitable excipients and carriers will be known to those skilled in the art. These may include solid or liquid carriers. Suitable liquid carriers include water or saline. The proteins of the composition may be formulated into an emulsion or alternatively they may be formulated in, or together with, biodegradable microspheres or liposomes.

Suitably the composition further comprises an adjuvant which stimulates the host's immune response. Particularly suitable adjuvants include Alhydrogel™, MPL+TDM™ and Freunds Incomplete Adjuvant.

In a preferred embodiment, the pharmaceutical composition further comprises an additional immunogenic polypeptide derived from Burkholderia pseudomallei. The additional polypeptide may be another selected from the group of proteins described herein or may be another protective antigen for Burkholderia pseudomallei.

In accordance with a third aspect of the invention, there is provided a nucleic acid which encodes a protein as described above. Particular examples include nucleic acids which encode the proteins of SEQ ID NO 4, 5 or 6. A nucleic acid which encodes protein of SEQ ID no 4 is preferred. Particular examples of other preferred nucleic acids are those of SEQ ID nos 1, 2 and 3 and, in particular, the nucleic acid of SEQ ID no 3 is more preferred.

Such nucleic acids can be used to produce the proteins described herein for use as medicaments. For instance they may be incorporated into an expression vector, which is used to transform an expression host, such as a prokaryotic or eukaryotic cell and, in particular, a prokaryotic cell such as Escherichia coli, using recombinant DNA technology as is well understood in the art. Such vectors, cells and expression methods form further aspects of the invention.

Alternatively, the nucleic acids can be used in “live” or “DNA” vaccines to deliver the protein to the host animal.

Thus, in a further aspect of the invention there is provided a nucleic acid, as described above for use as a medicament.

When used in this way, the nucleic acids will suitably be included within an expression vector such as a plasmid, and incorporated into a pharmaceutical composition. Thus, yet a further aspect comprises a pharmaceutical composition comprising a nucleic acid as described above in combination with a pharmaceutically acceptable carrier.

Other carriers that are suitable for use in the immunogenic composition include vectors for the delivery of the polypeptide, such as viral vectors (such as vaccinia or adenovirus), bacterial vectors (such as Salmonella) or plasmids. These vectors will allow expression of the polypeptide encoded by the nucleic acid within the host animal.

Suitable viral or bacterial vectors advantageously comprise human or animal gut colonising organisms that have been transformed using recombinant DNA to enable them to express the polypeptide or a protective epitopic part of the polypeptide. Salmonella-based vectors are particularly suitable but other live vectors are known in the art. Alternatively the composition may include so called naked DNA vaccines, wherein the nucleic acid such as DNA which encodes the required polypeptide is included in a plasmid.

The composition of the present invention may be used as vaccine against infections caused by Burkholderia species, such as melioidosis and glanders. The vaccine may be administered prophylactically to those at risk of exposure to Burkholderia mallei or Burkholderia pseudomallei or may be administered as a therapeutic treatment to persons who have already been exposed to these pathogens.

The route of administration of the vaccine may be varied depending on the formulation of the polypeptides of the composition. The composition may be suited to parenteral administration (including intramuscular, subcutaneous, intradermal, intraperitoneal and intravenous administration) but may also be formulated for non-parenteral administration (including intranasal, inhalation, oral, buccal, epidermal, transcutaneous, ocular-topical, vaginal, rectal administration).

In another aspect, the invention provides the use of a protein as described above in the preparation of a medicament for prophylactic or therapeutic vaccination against Burkholderia mallei or Burkholderia pseudomallei.

According to a further aspect the present invention relates to an antibody raised against the polypeptides described above, or a binding fragment thereof. Since the polypeptides are immunogenic, they are capable of inducing an immune response in a mammal to which they are administered. Antibodies may be raised in-vivo against the complete polypeptides, or they may be raised against suitable epitopic fragments of the polypeptides using conventional methods.

Binding fragments include Fab, F(ab′)2, Fc and Fc′.

Antibodies may be polyclonal or monoclonal. Hybridoma cell lines which generate monoclonal antibodies of this type form a further aspect of the invention.

Antibodies themselves, for example in the form of sera comprising such antibodies may be useful in the passive vaccination and/or treatment of compromised individuals.

According to a yet further aspect of the invention there is provided a method of protecting a human or animal body from the effects of infection with Burkholderia mallei or Burkholderia pseudomallei comprising administering to the body a vaccine comprising a polypeptide or a pharmaceutical composition as described above. The polypeptide is capable of inducing a protective immune response in a mammal to which it is administered and the ability to elicit an effective immune response may be provided by an epitopic fragment or a variant of said protein. Particular examples of suitable proteins include those described above. It is preferred that the protein is LolC (SEQ ID no.4) or a protective fragment or a protective variant thereof.

The polypeptide may be administered to the body by means of a “live” or DNA vaccine as described above.

The polypeptide utilised in the above method (and also present in the composition as described above) may be isolated from a suitable subspecies of Burkholderia pseudomallei, such as Burkholderia pseudomallei strain K96243 or, alternatively, it may be expressed in recombinant form and purified as appropriate as described as described in the examples herein.

The invention will now be particularly described by way of example with reference to the accompanying diagrammatic drawings where:

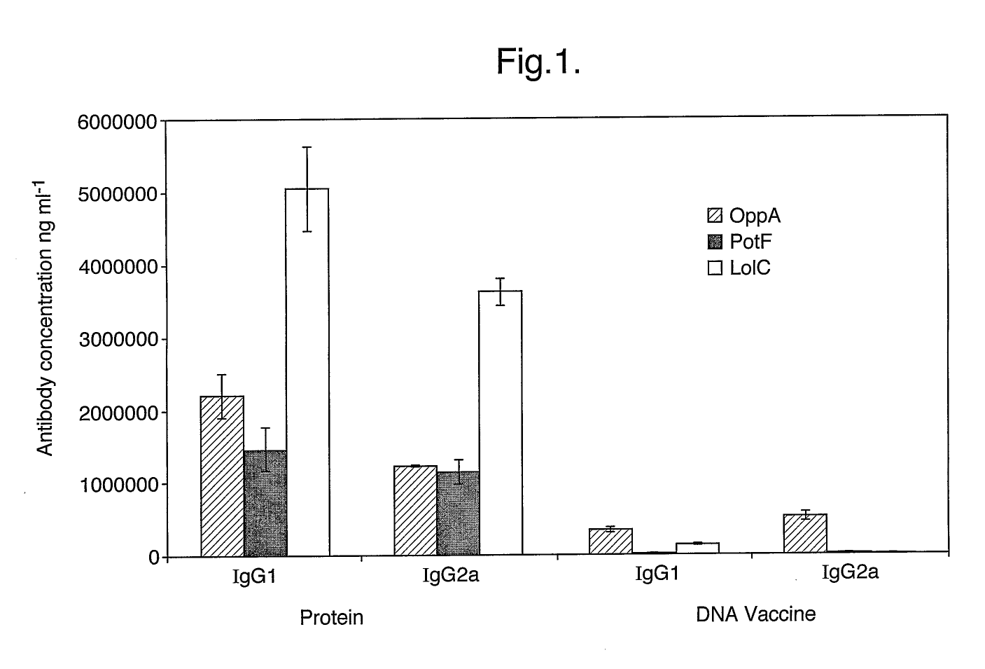

FIG. 1 shows the IgG1 and IgG2a response in mice to immunisation with PotF, LolC and OppA proteins and also the response with DNA vaccines prepared by transformation of the genes encoding said proteins.

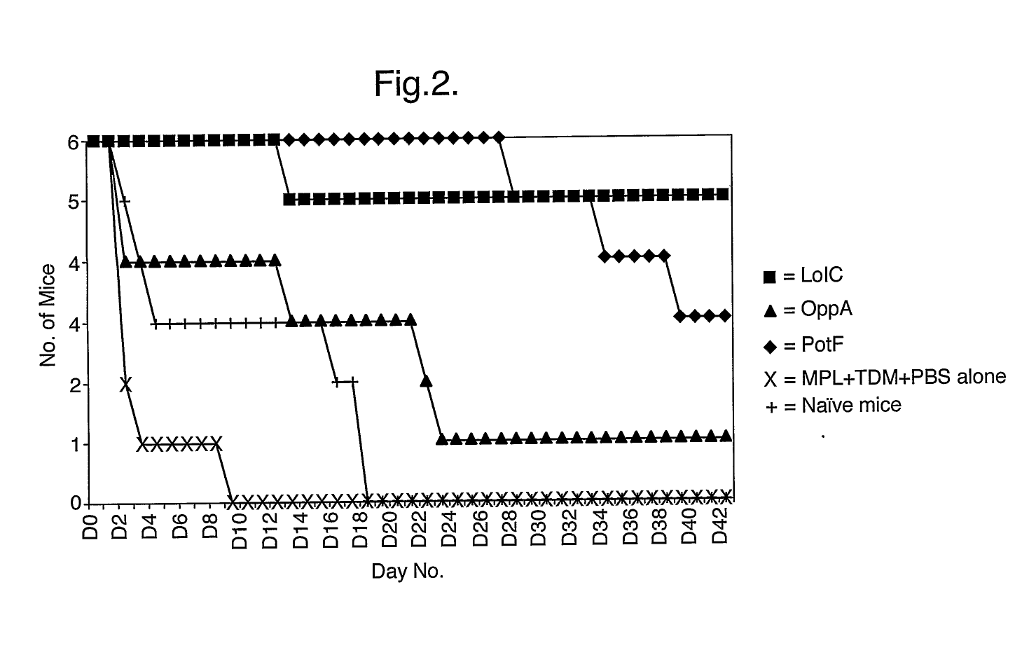

FIG. 2 shows survival curves for groups of six mice immunised with purified recombinant LolC, Oppa and PotF, control samples and naive mice which were subsequently challenged with approximately 4×10⁴ CFU of Burkholderia pseudomallei K96243.

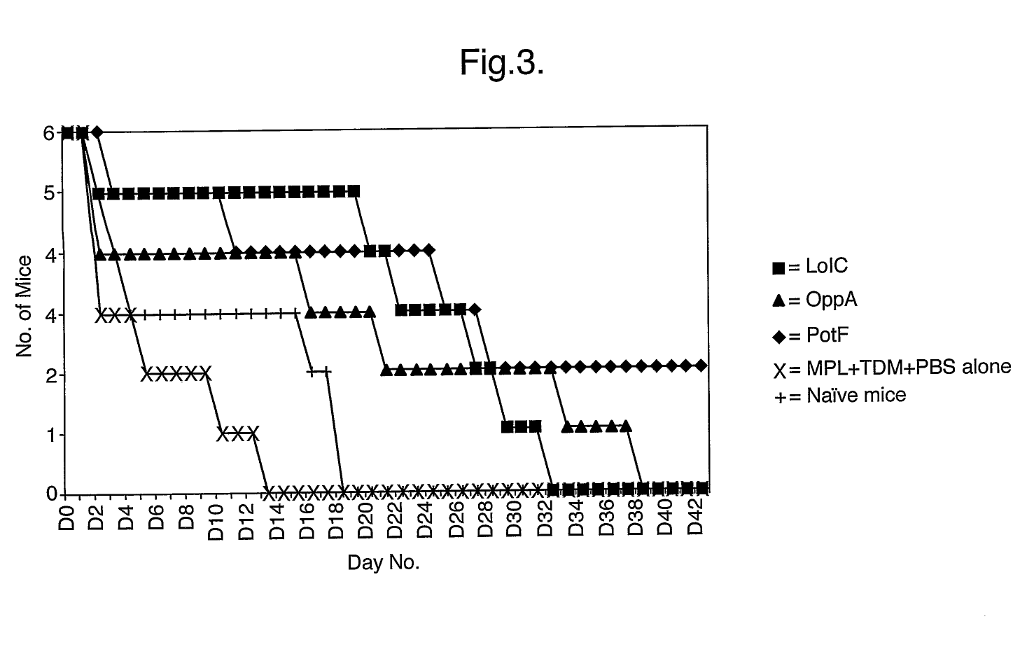

FIG. 3 shows survival curves for groups of six mice immunised with DNA vaccines encoding LolC, Oppa and PotF, control samples and naive mice which were subsequently challenged with approximately 4×10⁴ CFU of Burkholderia pseudomallei K96243.

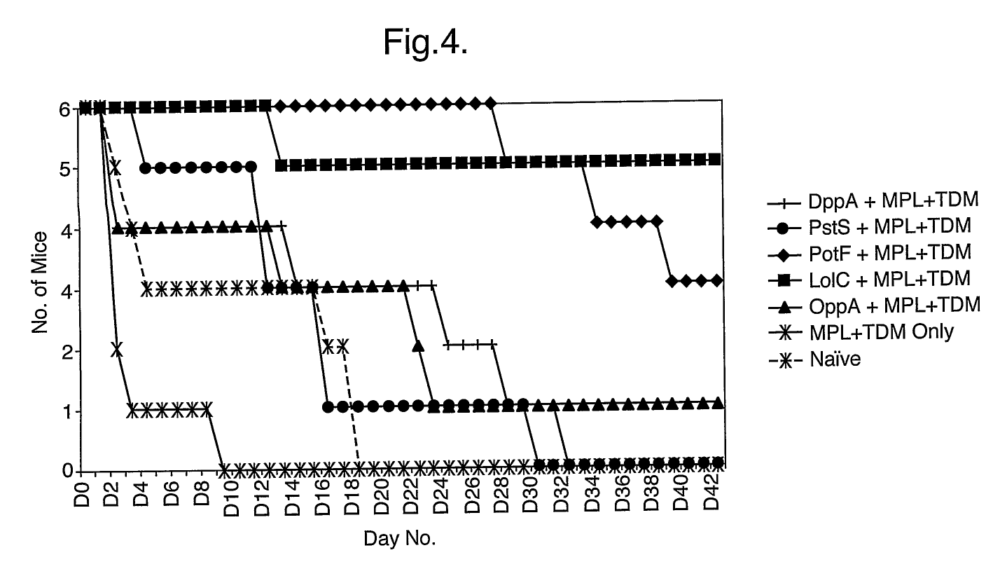

FIG. 4 shows survival curves for groups of 6 mice immunised with purified recombinant ABC system proteins (DppA, PstS, PotF, LolC, OppA) together with MPL+TDM adjuvant and naive mice that were subsequently challenged with approximately 4×10⁴ CFU of Burkholderia pseudomallei K96243

### BACTERIAL STRAINS, MEDIA AND CULTURE CONDITIONS

Escherichia coli TOP10F′ cells (Invitrogen Ltd. Paisley, UK) were used for cloning experiments and E. coli BL21 (DE3) pLysS cells (Invitrogen Ltd. Paisley, UK) were used for protein expression studies. The Burkholderia spp. strains from which purified DNA was provided are listed in Table 1. Burkholderia pseudomallei K96243 was used to challenge all mice. E, coli strains were cultured in Luria broth (L-broth) shaking at 180 rpm or on Luria agar (L-agar) plates at ACDP containment level 2 conditions. L-broth consisted of 1% (w/v) Bacto tryptone, 0.5% (w/v) Bacto yeast extract, and 0.5% (w/v) sodium chloride in distilled water. L-agar was prepared by the addition of 2% (w/v) Bacto agar to L-broth. Luria media was supplemented with ampicillin and/or chloramphenicol when appropriate. B. pseudomallei strains were cultured on nutrient agar or statically in nutrient broth at ACDP containment level 3 conditions. All bacteria were cultured at 37° C. for 18 hours unless otherwise stated.

TABLE 1
The presence of genes encoding ABC system
proteins in Burkholderia strains.

| | Gene | | |
|---|---|---|---|
| Burkholderia strain | oppA | potF | lolC |
| B. pseudomallei E38 | ✓ | ✓ | ✓ |
| B. pseudomallei 44 | ✓ | ✓ | ✓ |
| B. pseudomallei 2889 | ✓ | ✓ | ✓ |
| B. pseudomallei Mal6 | ✓ | ✓ | ✓ |
| B. pseudomallei NCTC 4845 | ✓ | ✓ | ✓ |
| B. pseudomallei 708A | ✓ | ✓ | ✓ |
| B. pseudomallei 576 | ✓ | ✓ | ✓ |
| B. pseudomallei 204 | ✓ | ✓ | ✓ |
| B. pseudomallei E8 | ✓ | ✓ | ✓ |
| B. pseudomallei K96243 | ✓ | ✓ | ✓ |
| B. mallei ATCC 10247 | ✓ | ✓ | ✓ |
| B. thailandensis E82 | ✓ | ✓ | ✓ |
| B. thailandensis E27 | ✓ | ✓ | ✓ |

✓ = present

### Identification and Selection of Potential Antigens

The Artemis genome viewer was used to locate PS00211, the ABC systems signature sequence, in the genome sequence of B. pseudomallei K96243 sequence (Holden, M. T. G. et al Proc. Nat. Acad. Sci. USA 101 (2004) 14240-14245). BLASTP searches were performed on the translated genes containing the signature sequence and on neighbouring genes in order to identify all ABC system genes in the B. pseudomallei K96243 sequence. ABC system proteins considered potentially involved in the virulence or immunogenicity of B. pseudomallei were selected for further study.

Screening of Different Burkholderia sp. Strains For Genes Encoding Selected Proteins

Oligonucleotide primer pairs were designed to amplify internal fragments of the genes encoding the selected proteins. Genomic DNA prepared from different to Burkholderia sp. strains was described previously (Table 1). PCR was performed using oligonucleotide primers and conditions detailed in Table 2. Amplified DNA was visualised by agarose gel electrophoresis to determine presence of genes encoding selected proteins in the different Burkholderia strains.

TABLE 2
Oligonucleotide primers and PcR conditions for
the amplification of internal fragments of
oppA, poTE and loLC for screening Burkholderia
strains

| | | PCR Conditions | | | | |
|---|---|---|---|---|---|---|
| | | Den. | Den. | Ann. | Ext. | Ext. |
| Gene | PCR Primers | ° C.^{a} | ° C. | ° C.^{b} | ° C.^{c} | ° C. |
| oppA | 5′-CGGCCGCGGA | 95 | 95 | 50 | 72 | 72 |
| | TTTCGTAT-3′F^{d} | | | | | |
| | 5′-GCAGCGCGGT | | | | | |
| | CTCGTCGTTC | | | | | |
| | T-3′R^{e} | | | | | |
| potF | 5′-ATCCAGGCCG | 96 | 96 | 66 | 72 | 72 |
| | GCATCTTCA-3′ | | | | | |
| | F | | | | | |
| | 5′-AGCGCGGCGT | | | | | |
| | CCTTGTTC-3′R | | | | | |
| lolC | 5′-AACTTTGAGG | 94 | 94 | 50 | 72 | 72 |
| | GCATGCTTACCGT | | | | | |
| | C-3′F | | | | | |
| | 5′-TCATTCATAG | | | | | |
| | CGCACCGCCTCCG | | | | | |
| | C-3′R | | | | | |

^{a} = Denaturing temperature ^{b} = Annealing temperature ^{c} = Extension temperature ^{d} = Forward primer ^{e} = Reverse primer

### Cloning and Expression

Amino acid sequences of the selected proteins were analysed using TMHMM v 2.0 (http://www.cbs.dtu.dk/services/TMHMM-2.0/) and SignalP (http://www.cbs.dtu.dk/services/SignalP/) to identify regions encoding transmembrane domains and signal peptides respectively. Oligonucleotide primers were designed to amplify DNA sequence encoding the non-membrane/non-signal peptide regions of the proteins. These gene truncates were amplified from B. pseudomallei K96243 genomic DNA by PCR using oligonucleotide primers and conditions listed in Table 3. The amplified gene fragments were cloned into the pCR®T7/NT-TOPO expression vector (Invitrogen Ltd. Paisley, UK), and the recombinant plasmids were transformed into Escherichia coli TOP10F′ cells (Invitrogen Ltd. Paisley, UK), according to manufacturer's instructions. Sequencing reactions were performed by MWG Biotech (Milton Keynes, UK) to ensure correct cloning and the incorporation of a N-terminal His₆-tag. Subsequently, E. coli BL21 (DE3) pLysS cells (Invitrogen Ltd. Paisley, UK) were transformed with the recombinant plasmids, according to manufacturer's instructions. Transformants were grown in culture and the addition of 1 mM Isopropyl-β-D-thiogalactoside (IPTG) was used to induce the expression of protein from the recombinant plasmids. Protein expression was confirmed by SDS-PAGE performed using Phastsystem gels and apparatus (Amersham Biosciences Chalfont-St Giles, UK) and visualised by PhastGel Blue R (Amersham Biosciences Chalfont-St Giles, UK). Expressed proteins were transferred to a membrane for detection by Western blot using transfer apparatus of the Phastsystem with the proteins detected with anti-His antibody and developed using 3,3′-Daminobenzidine (DAB) (Sigma-Aldrich Co. Ltd., Poole, UK).

TABLE 3

Oligonucleotide primers and PCR conditions for the amplification of
non-signal peptide/non-membrane regions of oppA, potF and lolC for
cloning experiments

PCR Conditions

| | | Den. | Den. | Ann. | Ext. | Ext. |
|---|---|---|---|---|---|---|
| Gene | PCR Primers | ° C.^{a} | ° C. | ° C.^{b} | ° C.^{c} | ° C. |
| oppA | 5′-AGGATCCTAATGTCGAATGTCACGCTC-′3 F | 96 | 96 | 61 | 72 | 72 |
| | BamHI | | | | | |
| | 5′-AAAGCTTTATTCAGTGCTTGATCAGGT-3′R | | | | | |
| | HindIII | | | | | |
| potF | 5′-AGAATTCTAATGAAGGATACGCAGCTC-′3 F | 96 | 96 | 49 | 72 | 72 |
| | EcoRI | | | | | |
| | 5′-AGCGGCCGCTATTCAGCGTCCCGATTTCAG-′3 R | | | | | |
| | NotI | | | | | |
| lolC | 5′-AGGATCCTAATGGTGCTGTAGGTGATG-′3 F | 96 | 96 | 55 | 72 | 72 |
| | BamHI | | | | | |
| | 5′-AAAGCTTTATTCACCGCTTCTCGATCT-′3 R | | | | | |
| | HindIII | | | | | |

### Protein Purification

To produce purified protein, E. coli BL21 (DE3) pLysS cells expressing recombinant protein were centrifuged at 10,000 rpm for 15 min. Pelleted cells were resuspended in Phosphate buffered saline (PBS) plus 300 μg m⁻¹ DNAase I and Complete EDTA-free protease inhibitor (Roche Diagnostics Ltd Lewes, UK). Cell suspensions were sonicated at 10 microns in 3×30 sec pulses and lysed cells were centrifuged at 15,000 rpm for 20 min. The supernatants were removed and sterilised through a 0.22 μm filter. Immobilised Metal Affinity Chromatography (IMAC) was carried out under the control of AKTA Fast Protein Liquid Chromatography (FPLC) (Amersham Biosciences Chalfont-St Giles, UK) and using Unicorn software version 4.0 (Amersham Biosciences Chalfont-St Giles, UK). Filtered supernatant was applied to a 1 ml His-Trap column (Amersham Biosciences Chalfont-St Giles, UK) with Start buffer (50 mM tris, 750 mM NaCl pH 7.5) and wash fractions were collected, according to manufacturers instructions. Recombinant His-tagged protein was eluted with elution buffer (50 mM Tris, 750 mM NaCl, 500 mM Imidazole, pH 7.5) and was collected in 1 ml fractions. Collected fractions were analysed for purified protein by SDS-PAGE. Fractions containing purified protein were dialysed in PBS to remove imidazole and purified protein was stored at −80° C.

### DNA Vaccine Construction and Preparation

Genes encoding selected proteins were sub-cloned from pCR®T7/NT-TOPO into the pVAX1 DNA vaccine vector using the restriction enzymes BamH1 and EcoR1 and DNA ligation using DNA rapid ligation kit (Roche Diagnostics Ltd Lewes, UK). E. coli TOP10F′ cells were transformed with recombinant pVAX1 vectors according to manufacturers instructions, and plasmids were purified from culture using a Qiagen Endofree Mega Prep kit (Qiagen, Crawley, UK). The correct sequence of the genes in the vector was determined by sequencing at MWG biotech (Milton Keynes, UK). DNA vaccine constructs were stored at −20° C.

Immunisation and Challenge with B. pseudomallei K96243

DNA vaccine constructs and purified proteins were produced for evaluation as candidate vaccines against B. pseudomallei. The concentrations of the proteins were ascertained by BCA assay (Pierce Biotechnologies) and purity was evaluated by SDS-PAGE and staining with PhastGel Blue R. Proteins were prepared for immunisation mixed 1:1 with MPL+TDM adjuvant (Sigma-Aldrich Co. Ltd., Poole, UK) and as 100 μg ml⁻¹ concentrations. DNA vaccine immunisations contained 100 μg of plasmid DNA in 0.25% (w/v) bupivicane hydrochloride (Astra pharmaceuticals, Kings Langley, UK) in 100 μl PBS. Groups of 6 female BALB/c mice (Charles River Laboratories Margate, UK) (5 to 6 weeks old) were used. Proteins were delivered by intraperitoneal (i.p.) injection of 100 μl of each protein in MPL+TDM® adjuvanton days 0, 14 and 28. DNA vaccines were injected intramuscularly (i.m.) with 50 μl of inoculum in each hind leg of each mouse on days 0 and 14. On day 28, mice immunised with DNA vaccines were boosted with a single i.p. immunisation of the corresponding purified protein at 100 μg ml⁻¹ concentrations. Blood was taken via the tail vein from each mouse on day 56. Blood samples were allowed to clot for 2-24 h at 4° C. and the centrifuged for 10 min at 13,000 rpm and the serum was stored at −20° C. until use. On day 70 the mice were challenged with approximately 4×10⁴ colony forming units (cfu) of B. pseudomallei K96243 via the i.p. route. Groups of age matched mice were left untreated, or given adjuvant only or PVAX1 only as controls as controls.

### Antibody Response Analysis

The LolC-specific, OppA-specific and PotF-specific IgG1 or IgG2a responses in blood samples removed from mice prior to challenge were determined by enzyme-linked immunosorbant assay. Briefly, microtitre plates were coated with 5 μg ml⁻¹ of recombinant protein in PBS over night. Three columns on each plate were coated with anti-IgG1 (fab) or anti-IgG2a (fab) in order to produce a standard curve for quantification of IgG1 or IgG2a concentration respectively. The plates were blocked using 2% (w/v) bovine serum albumin in PBS (BSA-PBS) for 1 h at 37° C. Plates were washed and serum samples diluted in BSA-PBS were serially diluted 1:2 down the plate in duplicate and incubated for 2 h at 37° C. IgG1 or IgG2a isotype standards were also serially diluted 1:2 down the plate wells coated with anti-IgG1 (fab) or anti-IgG2a (fab) respectively. Three washes with PBS plus 0.05% (v/v) Tween 20 were carried out between each step. Goat anti-mouse IgG1 or IgG2a horseradish peroxidase conjugates diluted in BSA-PBS were added and after a further 2 h at 37° C., bound antibody was visualised by adding ABTS substrate. The plates were incubated at room temperature for 20 min before measuring OD₄₁₄ₙₘ. Serum antibody concentration was calculated in ng ml⁻¹ using the standard curve (Ascent™ software version 2.4.2 Thermo Electron Corporation, Basingstoke, UK).

### Results

### Proteins Selected as Potential Vaccine Antigens

Genes encoding predicted ABC system proteins in the B. pseudomallei K96243 genome sequence were identified using bioinformatic tools. Three protein components of three different predicted ABC systems were selected for further investigation as potential antigens through their predicted roles in virulence or immunogenicity of B. pseudomallei.

OppA (BPSS2141) is a putative periplasmic binding protein component of the oligopeptide (Opp) ABC transporter system. In E. coli, OppA handles peptides that range widely in number of amino acids in length. The OppA protein is thought to be involved in the intracellular survival of Listeria monocytogenes in the macrophage and for bacterial growth in mice. This effect is probably due to either the uptake of peptides which are protective against the actions of the phagosome, or the uptake of peptides that bring about a modulation in the expression of proteins, thereby affording greater protection.

PotF (BPSL1555) is a putative periplasmic binding protein of a polyamine uptake system. In E. coli, PotF is a periplasmic binding protein of a putrescine-specific ABC transporter system. The polyamines putrescine, spermidine and spermine are required for cell growth in eukaryotes and prokaryotes. PotF may have inhibitory effects upon potFGHI operon expression since PotD, a homologue of PotF, is thought to have an inhibitory effect on potABCD operon expression. As cellular accumulation of polyamines rises, a precursor of PotD acts as a transcriptional regulator, reducing the expression of polyamine uptake systems. The periplasmic location of PotF indicates that it may be presented to the host immune system and in this context may constitute an antigen.

LolC (BPSL2277) is a putative inner membrane protein of the LolCDE ABC transporter. In E. coli this system provides the energy for the transport of lipoproteins directed to the outer membrane, which are released from the inner membrane in complex with LolA, a periplasmic chaperone. Lipoproteins are synthesised in the cytoplasm of a bacterial cell and are exported to either the inner or the outer membrane for incorporation. The LolCDE complex is an essential ABC transporter for E. coli and the sole apparatus mediating the release of outer membrane lipoproteins from the inner membrane.

### Genes Encoding Selected Proteins are Detected in a Range of Burkholderia Strains

The genomic DNA of 11 different strains of Burkholderia sp. was screened by PCR for the presence of the genes encoding the selected proteins using oligonucleotide primer pairs designed to amplify short internal fragments of the genes. OppA, PotF and LolC were found to be present in strains of B. pseudomallei, B. thailandensis and B. mallei. This evidence suggests that any future vaccine or therapeutic based on these proteins is likely to be protective against all strains of Burkholderia sp.

### Production of Purified Recombinant OppA, PotF and LolC Proteins

The predicted non-membrane/non-signal peptide regions of the selected proteins were identified using TMHMM v 2.0 (http://www.cbs.dtu.dk/services/TMHMM-2.0/) and SignalP (http://www.cbs.dtu.dk/services/SignalP/). B. pseudomallei K96243 DNA encoding the non-membrane/non-signal peptide regions of the selected proteins (FIGS. 1a, b and c) was amplified by PCR and cloned into the expression vector pCR®T7/NT-TOPO. This vector was used to allow expression of the proteins as fusions to a His-tag, enabling purification by His-Trap™. E. coli BL21 (DE3) pLysS cells transformed with recombinant plasmids were induced to express the selected proteins which were subsequently purified by Immobilised Metal Affinity Chromatography. Following SDS-PAGE analysis, proteins of approximately 57 KDa, 38 KDa, and 24.5 KDa could be visualised (not shown) corresponding to the predicted molecular masses of OppA, PotF and LolC respectively. The purified proteins all reacted with anti-His monoclonal antibody in a Western blot (data not shown).

### Construction of DNA Vaccines

To further investigate the potential of the selected proteins as vaccine antigens, DNA vaccines encoding the selected proteins were produced as an alternate vaccine delivery technology. The DNA vaccines were constructed by sub-cloning the genes encoding non-membrane/non-signal peptide regions of OppA, LolC and PotF from pCR®T7/NT-TOPO into a pVAX1 vector without the inclusion of a His-Tag. The insertion of the genes into the pVAX1 vector was confirmed by restriction digest analysis and sequencing.

Immunisation of mice with OppA, PotF and LolC

The non-membrane/non-signal peptide protein antigens, OppA, PotF and LolC and the corresponding DNA vaccines were used to immunise groups of mice. Briefly, mice were either administered with 3 doses of recombinant protein or 2 doses of DNA vaccine followed by 1 dose of recombinant protein (DNA prime/protein boost). Sera were collected via the tail vein 28 days after the final immunisation, and the sera from each group were pooled. The sera were analysed for specific antibody responses to each protein by ELISA (FIG. 3). Specific IgG1 and IgG2a responses to OppA, PotF and LolC could be detected in mice given the recombinant proteins. In comparison, only low levels of antibody could be detected in mice given the OppA and LolC DNA vaccines and mice given the PotF DNA vaccine had undetectable antibody responses. The recombinant proteins induced similar levels of IgG1 and IgG2a with a slight bias towards an IgG1 antibody (Th2 type) response. Mice immunised with PotF and OppA protein antigens showed greater IgG1 and IgG2a responses to the antigens in comparison to mice given the naïve control and adjuvant only controls. Mice immunised with LolC protein antigen produced greater IgG1 and IgG2a response than both the control mice and those immunised with PotF and OppA. The sera analysis indicates that the protein antigens, especially LolC, are potent immunogens, particularly in respect to IgG antibody response. DNA vaccines appear to be less effective at initiating an IgG antibody response. This is not unexpected, since it is known that DNA vaccines are effective stimulators of cellular immune responses.

Protection Afforded Against B. pseudomallei

The mice immunised with recombinant OppA, PotF or LolC, or given corresponding DNA prime/protein boost immunisations were challenged with approximately 4×10⁴ cfu B. pseudomallei K96243 (equivalent to approximately 40 MLD) via the i.p. route and the health status of the mice was monitored for 42 days. Control mice given adjuvant or pVAX1 DNA only, or left untreated, all died by day 19 post-infection. However, protection afforded against B. pseudomallei infection could be observed in mice given either the proteins (FIG. 1) or DNA prime/protein boost (FIG. 2). Mice immunised with OppA protein demonstrated an extended time to death in comparison to both the naïve control (p=<0.0001) and adjuvant only control mice (p=<0.0001) and also included 1/6 mouse that survived to the end of the experiment (FIG. 2). An extended time to death was also observed in the group of mice immunised with the PotF protein in comparison to both the naïve control (p=<0.0001) and adjuvant only control mice (p=<0.0001), and 3/6 mice from this group survived to the end of the experiment. Immunisation with LolC protein had the greatest effect on survival, where 5/6 mice from this group survived to the end of the experiment. In comparison, mice immunised with DNA vaccines and given a protein boost, were not as well protected as those that received protein antigens. However, all the immunised mice demonstrated a significant extended time to death when compared to both the naïve control (p=<0.0001) and adjuvant only control mice (p=<0.0001). In addition, of the mice immunised with PotF DNA vaccine prime/boost, 2/6 survived to the end of the experiment. The results of this study indicate that the selected protein antigens, delivered either as purified protein in adjuvant or in a DNA vaccine prime/protein boost regime may constitute protective antigens against B. pseudomallei infection. Since the highest level of protection afforded was by the LolC purified recombinant protein it is possible that IgG antibody responses are important in protection. The high IgG concentrations present may prevent colonisation by B. pseudomallei either through the process of opsonisation or direct neutralisation of the pathogen. However, an extended time to death was observed in all DNA vaccine groups and 2 mice immunised with the DNA vaccine encoding PotF survived the experiment. Since the DNA vaccines were ineffective at inducing IgG responses, this indicates that protection was mediated through an alternative immune mechanism. Further work will be required understand the mechanisms of protection afforded.

The results described herein identify OppA, PotF and LolC proteins of B. pseudomallei as protective antigens. This is the first known demonstration of any protein providing protection against B. pseudomallei infection. Combinations of these proteins could be used together to enhance the protection observed with individual proteins. Since the genes encoding the protective proteins have been detected in a range of B. pseudomallei strains, the antigens should be protective against all B. pseudomallei strains. Also, as the genes have been detected in a B. mallei strain, the proteins may also protect against glanders, the disease resulting from B. mallei infection. Therefore, these proteins should cross-protect against both melioidosis and glanders. Furthermore, since homologues of the protective proteins can be found in a range of bacteria, OppA, PotF and LolC may provide protection against a range of bacterial infections and as a result could be used as a generic vaccine. As genes encoding the protective proteins are not present in human or mouse genomes it is unlikely that a vaccine based on these proteins would have a deleterious affect. Since it has also been observed that antibody response maybe important in protection, it is possible that anti-sera raised against proteins could be used therapeutically, with the potential as use in generic therapy.

1. A protein expressed by an ATP-binding cassette system gene of Burkholderia pseudomallei or a fragment or a variant of the protein, wherein the protein, fragment, or variant is capable of producing a protective immune response against Burkholderia mallei or Burkholderia pseudomallei.

2. The protein according to claim 1, selected from the group consisting of OppA, PotF and LolC.

3. (canceled)

4. The protein of claim 1 having an amino acid sequence selected from the group consisting of SEQ ID NO. 4, SEQ ID NO. 5 and SEQ ID NO. 6.

5. (canceled)

6. A pharmaceutical composition comprising a protein expressed by an ATP-binding cassette system gene of Burkholderia pseudomallei or a fragment or a variant of said the protein, wherein the protein, fragment, or variant is capable of producing a protective immune response against Burkholderia mallei or Burkholderia pseudomallei, in combination with a pharmaceutically acceptable carrier, excipient, adjuvant or combination thereof.

7. The pharmaceutical composition according of claim 6 wherein the protein, fragment or variant is encoded by a nucleic acid molecule having a nucleic acid sequence selected from the group consisting of SEQ ID NO. 1, SEQ ID NO. 2 and SEQ ID NO. 3.

8. The pharmaceutical composition according of claim 6 wherein the protein has an amino acid sequence selected from the group consisting of SEQ. ID NO. 4, SEQ ID NO. 5 or SEQ ID NO. 6.

9. (canceled)

10. (canceled)

11. A nucleic acid which encodes the protein of claim 1.

12. The nucleic acid of claim 11 having a nucleic acid sequence selected from the group consisting of SEQ ID NO. 1, SEQ ID NO. 2 and SEQ ID NO. 3.

13. (canceled)

14. A method of producing a protective immune response against a Burkholderia infection in a mammal comprising administering to the mammal the pharmaceutical composition of claim 6.

15. The method of claim 14 wherein the Burkholderia infection is caused by a Burkholderia mallei, Burkholderia pseudomallei or Burkholderia thailandensis infection.

16. (canceled)

17. (canceled)

18. An antibody raised against the protein, fragment or variant of claim 1.

19. (canceled)

20. (canceled)

21. A polypeptide having an amino acid sequence of SEQ ID NO. 5, or a fragment or a variant of the polypeptide wherein the polypeptide, fragment or variant is capable of producing a protective immune response against Burkholderia pseudomallei and/or Burkholderia mallei.

22. A polypeptide having an amino acid sequence of SEQ ID NO. 6, or a fragment or a variant of the polypeptide wherein the polypeptide, fragment or variant is capable of producing a protective immune response against Burkholderia pseudomallei and/or Burkholderia mallei.
